(19) United States
(12) Patent Application Publication
(10) Pub. No.: US 2026/0311061 A1
(43) Pub. Date: Oct. 8, 2026

(54) METHOD FOR CONSTRUCTING MACHINE LEARNING MODEL FOR RECOMMENDING AIRWAY OPENING POSTURE ANGLE

(71) Applicant: CHONGQING HAOHUXI MEDICAL DEVICES CO., LTD., Chongqing (CN)

(72) Inventors: Xiangmei YANG, Chongqing (CN); Li TAN, Chongqing (CN); Jinjin GUO, Chongqing (CN); Yun LIAO, Chongqing (CN); Yan LUO, Chongqing (CN)

(73) Assignee: CHONGQING HAOHUXI MEDICAL DEVICES CO., LTD., Chongqing (CN)

(21) Appl. No.: 19/638,139

(22) Filed: Apr. 3, 2026

(30) Foreign Application Priority Data
Apr. 3, 2025 (CN) 2025104167948

(51) Int. Cl. G06N 20/00; A61B 1/00; A61B 1/267; A61B 34/10

(52) U.S. Cl. CPC G06N 20/00; A61B 1/000096; A61B 1/267; A61B 34/10; A61B 2034/107

(57) ABSTRACT

Disclosed is a method for constructing a machine learning model for recommending an airway opening posture angle. The method includes collecting independent variable data affecting a glottis exposure effect when opening a patient airway, conducting an airway opening experiment, taking a posture angle corresponding to an optimal glottis exposure image as an optimal posture angle, performing single-factor analysis on the independent variables, and screening out statistically significant independent variable data as candidate prediction variables. The method further includes preselecting machine learning models, training and verifying the models, evaluating using AUC values, and selecting a model with a highest AUC value as a prediction model for recommending the optimal airway opening posture angle. The obtained prediction model accurately recommends the optimal airway opening posture angle to expose a glottis of the patient for medical personnel, reducing or avoiding a risk of glottis exposure difficulty in bronchoscopy diagnosis and treatment.

### CROSS-REFERENCE TO RELATED APPLICATIONS

The application claims priority to Chinese patent application No. 2025104167948, filed on Apr. 3, 2025, the entire contents of which are incorporated herein by reference.

### TECHNICAL FIELD

The present disclosure relates to the technical field of medicine, particularly to a method for recommending an optimal airway opening angle.

### BACKGROUND

Difficulty in glottis exposure refers to a situation where a glottis structure cannot be smoothly observed due to various reasons during endotracheal intubation or laryngoscopy. According to research at home and abroad, an incidence rate of difficulty in glottis exposure under a laryngoscope is 1.5% to 13.0%. A restricted glottis visual field can often increase risks of airway mucosal injury, aspiration, and the like in a patient, and may even lead to intubation failure or prolonged intubation time, thereby causing severe hypoxemia in the patient and resulting in irreversible brain damage or death.

Fully and effectively opening an airway to achieve optimal glottis exposure can reduce or avoid occurrence of difficulty in glottis exposure to a certain extent, thereby reducing operation difficulty and risk. Clinically, a primary step of bronchoscopy diagnosis and treatment, same as endotracheal intubation, is opening the airway to expose a glottis. However, during an actual operation process, since a bronchoscope has a slender diameter and is easy to pass through, issues such as opening the airway and exposing the glottis are often easily ignored.

Bronchoscopy is a diagnosis and treatment method in which a bronchoscope is inserted into a respiratory tract through an oral cavity or a nasal cavity of a patient, and reaches positions such as a trachea and a bronchus through the glottis to discover or treat lesions. Due to characteristics of simple operation, safety, and effectiveness, bronchoscopy is widely applied in diagnosis and treatment of respiratory system related diseases. However, as an airway invasive operation, even if the diameter is slender, the patient may still experience symptoms such as strong choking cough, airway spasm, nausea, and vomiting during the bronchoscopy diagnosis and treatment. In recent years, with in-depth development of bronchoscopy technology, an operation time and difficulty of bronchoscopy gradually increase, and higher requirements are placed on patient compliance, operation safety, and comfort. Therefore, the 2019 edition of “Guidelines for Application of Diagnostic Flexible Bronchoscopy in Adults” points out that, if there is no contraindication, a sedative should be routinely given to a bronchoscopy patient before surgery. Therefore, in current clinical practice, except for relatively complex rigid bronchoscopy surgeries adopting general anesthesia, routine bronchoscopy diagnosis and treatment are performed under local anesthesia combined with sedation and analgesia. Benzodiazepine midazolam, as a preferred sedative drug for bronchoscopy, is often used in combination with opioids, propofol, dexmedetomidine, and the like to achieve a better sedative effect. However, during a diagnosis and treatment process, sedative or anesthetic drugs can affect nerve conduction and muscle contraction functions, causing muscles of a soft palate and a throat to relax and lose normal muscle tension. A backward movement of a tongue base can block a posterior pharyngeal wall, and a drooping of the soft palate blocks a passage between a nasopharynx and an oropharynx, narrowing or even blocking the airway, thereby causing upper respiratory tract obstruction and affecting glottis visual field exposure. Moreover, according to physiological structure characteristics of the patient, a degree of glottis visual field exposure varies.

Good airway opening can reduce difficulty and risk brought by bronchoscopy diagnosis and treatment, and is an important link to ensure smooth progress of the diagnosis and treatment. Among numerous factors affecting a degree of airway opening during bronchoscopy diagnosis and treatment, posture management is particularly important. However, there is currently no optimal posture recommendation for bronchoscopy diagnosis and treatment in guidelines at home and abroad. Therefore, clinically, a supine position without a pillow is often taken, and artificial methods such as a head-tilt chin-lift method, a head-tilt neck-lift method, and a jaw-thrust method are used to fully extend a head and a neck of the patient to open the airway. Although conventionally taking the supine position without a pillow supplemented by the artificial methods to open the airway is simple and convenient, during an actual operation process, due to gravity, a tongue base of a sedated or anesthetized patient in a supine position state falls backward to block a pharyngeal cavity, and may even press against an epiglottis to cover the glottis. Moreover, due to differences in physiological structures of patients and airway opening techniques of operators, situations such as an insufficient airway opening angle, excessive opening, or no opening easily occur, thereby resulting in difficulty in glottis exposure. Once the above situations occur, an airway protection mechanism of the patient may be affected, and secretions and vomitus in an oral cavity and a throat are easily aspirated into the airway by mistake, causing severe complications such as aspiration pneumonia and asphyxia. Due to a restricted glottis visual field, repeatedly opening the airway or making multiple attempts to insert a scope may stimulate and damage the glottis and surrounding tissues, thereby causing vocal cord edema, laryngeal mucosal bleeding, and even causing severe damage such as laryngeal cartilage fracture. The severe damage not only increases suffering of the patient, but also may affect postoperative vocalization function and airway patency of the patient, increasing a risk of airway stenosis. In addition, hypoxia and carbon dioxide retention caused by prolonged bronchoscopy operation time, as well as stress reactions during an operation process, stimulate a sympathetic nervous system of a body, causing arrhythmia, leading to diagnosis and treatment failure or a need for a secondary diagnosis and treatment, thereby increasing an incidence rate of discomfort and complications after the bronchoscopy diagnosis and treatment.

Accordingly, recommending an optimal airway opening angle for the patient to achieve optimal glottis exposure is an effective measure to reduce discomfort and complications after bronchoscopy diagnosis and treatment. In a prior invention patent with an application number of 2022111651871, an inventor disclosed a control method of an automatic airway opening posture adjustment device. Through an experiment of opening the airway of the patient conducted by using the control method, an observation is made that when the airway is opened at states of 3 posture angles being 90°, 95°, and 100°, CL (Cormack-Lehane) grades of a vast majority of patients are Grade I or Grade II, indicating that the control method can effectively open the airway of the patient and obtain a good glottis visual field, which is beneficial for a bronchoscope to smoothly pass through the glottis to enter a lower respiratory tract and continuously maintain airway patency.

However, due to individual differences among patients, when opening the airway of the patient, which posture angle among 90°, 95°, and 100° is the optimal posture angle for allowing the glottis of the patient to be exposed is unknown to medical personnel. If every angle is tried once, valuable treatment time is consumed. Therefore, constructing a prediction model capable of accurately predicting an optimal airway opening posture angle for a patient is a technical problem urgently needing to be solved at present.

### SUMMARY

A Accordingly, an objective of the present disclosure is to provide a method for constructing a machine learning model for recommending an airway opening posture angle, to solve a technical problem of providing a prediction model for medical personnel to achieve accurate prediction of an optimal airway opening posture angle of a patient.

The method for constructing the machine learning model for recommending the airway opening posture angle of the present disclosure includes the following steps:
collecting independent variable data possibly affecting a glottis exposure effect when an airway of the patient is opened, each patient corresponding to a group of independent variable data;
conducting an airway opening experiment on the patient corresponding to each group of the independent variable data, collecting glottis exposure images of the patient respectively at three preselected posture angles being 90°, 95°, and 100° in the experiment, performing laryngoscope exposure grading on a glottis exposure state in the images, and selecting an optimal posture angle making the patient be in an optimal glottis exposure state from the three preselected posture angles according to a laryngoscope exposure grading situation; and saving the optimal posture angle and the corresponding independent variable data;
performing an assignment processing on count independent variable data in each group of the saved independent variable data, performing an original value retention processing on measurement independent variable data in each group of the saved independent variable data, performing an assignment processing on the optimal posture angle corresponding to each group of the saved independent variable data, an assignment of the optimal posture angle serving as an outcome dependent variable, then inputting data subjected to aforementioned processing into a statistical package for social sciences to perform a single-factor analysis on various independent variables, and screening out independent variables with statistical differences according to a single-factor analysis result;
selecting out independent variable data with statistical significance in each group of the independent variable data according to an independent variable screening result, and configuring a data set by using the independent variable data with statistical significance and the outcome dependent variable;
preselecting several machine learning models configured to recommend the optimal airway opening posture angle, dividing the data set into a training set and a verification set, and respectively training and verifying each machine learning model by using data in the training set and the verification set; and
aiming at various machine learning models obtained through training and verification, evaluating the various machine learning models by using average AUC values classified for the three preselected posture angles being 90°, 95°, and 100°, and selecting a machine learning model with a highest average AUC value as a prediction model for recommending the optimal airway opening posture angle.

Optionally, in step 2), a method for selecting the optimal posture angle making the patient be in the optimal glottis exposure state from the three preselected posture angles according to the laryngoscope exposure grading situation includes:
according to the laryngoscope exposure grading situation of glottis exposure, if there is a preselected posture angle with a laryngoscope exposure grade below grade II, taking a preselected posture angle making the laryngoscope exposure grade lowest as the optimal posture angle, and if there are two or more aforementioned preselected posture angles, selecting a preselected posture angle with a minimum angle value as the optimal posture angle;
if laryngoscope exposure grades corresponding to the three preselected posture angles are all above grade III, an optimal posture angle corresponding to the group of independent variable data does not exist, and deleting the group of independent variable data.

Optionally, the independent variable data collected in step 1) includes: gender, age, body mass index, modified Mallampati score, upper lip bite test grade, head and neck mobility, mouth opening degree, neck circumference, hyomental distance, thyromental distance, sternomental distance, ratio of height to thyromental distance, ratio of neck circumference to thyromental distance, and ratio of height to hyomental distance;
an acquisition method of the upper lip bite test grade is: the patient sits upright, a mandible is extended forward as much as possible, and lower incisors are used to bite an upper lip as much as possible, if an upper lip line is exceeded, a grade is grade I; if the lower incisors are lower than the upper lip line, the grade is grade II; and if the lower incisors cannot bite the upper lip, the grade is grade III;
the head and neck mobility refers to an angle formed by maximally flexing a neck forward to make a mental prominence lean against a sternal notch, and a maximal head back-tilt position;
the mouth opening degree refers to a distance between upper and lower central incisors when the patient is in a neutral sitting position and opens a mouth to a maximum extent;
the hyomental distance refers to a distance from an anterior border of a hyoid bone to a mental protuberance of the mandible;
the thyromental distance refers to a linear distance from a superior thyroid notch to a submental angle of the mandible when the patient is in a maximal head back-tilt state and the mouth is closed;
the sternomental distance refers to a linear distance from a suprasternal notch to the submental angle of the mandible when the patient closes the mouth and the head and neck are in a maximal back-extension position;
the ratio of height to thyromental distance refers to a ratio of height to the thyromental distance;
the ratio of neck circumference to thyromental distance refers to a ratio of the neck circumference to the thyromental distance;
the ratio of height to hyomental distance refers to a ratio of the height to the hyomental distance.

Optionally, an acquisition method of the modified Mallampati score is: the patient takes a sitting position, a head is in a neutral position, the mouth is opened and a tongue is extended to a maximum extent as much as possible without phonation, and grading is performed according to visibility of an oropharyngeal structure; grade I: a soft palate, a palatopharyngeal arch, and a uvula are visible; grade II: the soft palate and the palatopharyngeal arch are visible, but the uvula is partially blocked by a tongue base; grade III: only the soft palate is visible; and grade IV: the soft palate is not visible.

Optionally, in step 3), the independent variables with statistical differences screened out according to the single-factor analysis result are age, modified Mallampati score, hyomental distance, thyromental distance, sternomental distance, ratio of height to thyromental distance, ratio of neck circumference to thyromental distance, and height hyomental distance.

Optionally, the machine learning models preselected in step 5) include logistic regression, a decision tree, and a random forest.

Optionally, in step 6), the random forest is selected as the prediction model for recommending the optimal airway opening posture angle.

Beneficial effects of the present disclosure:
the method for constructing the machine learning model for recommending the airway opening posture angle of the present disclosure is provided, a prediction model obtained by the method is capable of accurately recommending the optimal airway opening posture angle for exposing the glottis of the patient for the medical personnel, and further capable of reducing or avoiding a risk of difficulty in glottis exposure in bronchoscopy diagnosis and treatment. Moreover, through predicting the posture angle for opening the airway of the patient in advance, consuming excessive diagnosis and treatment time by a process of opening the airway can be avoided.

### BRIEF DESCRIPTION OF DRAWINGS

FIG. 1 is a screenshot of results of a single-factor analysis generated using SPSS.

### DETAILED DESCRIPTION OF THE EMBODIMENTS

The present disclosure is further described below with reference to the accompanying drawings and examples.

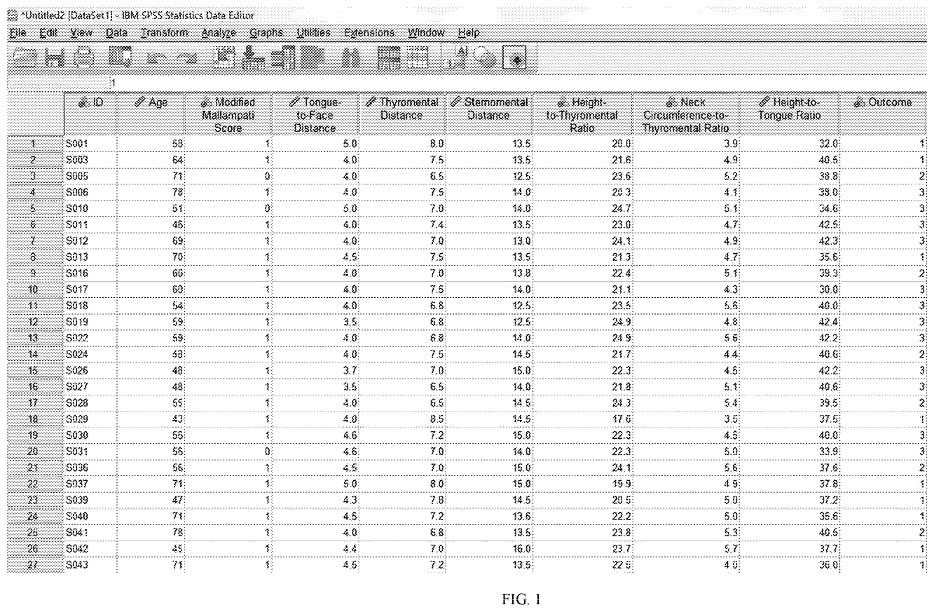

As shown in the FIG. 1, a method for constructing a machine learning model for recommending an airway opening posture angle in the example includes the following steps:
1) collecting various independent variable data possibly affecting a glottis exposure effect when an airway of a patient is opened, each patient corresponding to a group of independent variable data.

The independent variable data collected in the example include: gender, age, body mass index, modified Mallampati score, upper lip bite test grade, head and neck mobility, mouth opening degree, neck circumference, hyomental distance, thyromental distance, sternomental distance, ratio of height to thyromental distance, ratio of neck circumference to thyromental distance, and ratio of height to hyomental distance.

A calculation formula of the body mass index (BMI) is: BMI=weight (kg)/height (m)2.

An acquisition method of the modified Mallampati score is: the patient takes a sitting position, a head is in a neutral position, a mouth is opened and a tongue is extended to a maximum extent as much as possible without phonation, and grading is performed according to visibility of an oropharyngeal structure; grade I: a soft palate, a palatopharyngeal arch, and a uvula are visible; grade II: the soft palate and the palatopharyngeal arch are visible, but the uvula is partially blocked by a tongue base; grade III: only the soft palate is visible; and grade IV: the soft palate is not visible.

An acquisition method of the upper lip bite test grade is: the patient sits upright, a mandible is extended forward as much as possible, and lower incisors are used to bite an upper lip as much as possible, if an upper lip line is exceeded, a grade is grade I; if the lower incisors are lower than the upper lip line, the grade is grade II; and if the lower incisors cannot bite the upper lip, the grade is grade III.

The head and neck mobility refers to an angle formed by maximally flexing a neck forward to make a mental prominence lean against a sternal notch, and a maximal head back-tilt position; and may be measured and obtained by using an angle ruler.

The mouth opening degree refers to a distance between upper and lower central incisors when the patient is in a neutral sitting position and opens the mouth to a maximum extent; and may be measured and obtained by using a straight ruler.

The neck circumference is measured and obtained by using a tape measure to closely stick to a lower edge of a laryngeal prominence and circle the neck once under a condition that the patient takes an upright sitting position and looks straight ahead horizontally.

The hyomental distance refers to a distance from an anterior border of a hyoid bone to a mental protuberance of the mandible, and may be measured and obtained by using the tape measure.

The thyromental distance refers to a linear distance from a superior thyroid notch to a submental angle of the mandible when the patient is in a maximal head back-tilt state and the mouth is closed; and may be measured and obtained by using the tape measure.

The sternomental distance refers to a linear distance from a suprasternal notch to the submental angle of the mandible when the patient closes the mouth and the head and neck are in a maximal back-extension position; and may be measured and obtained by using the tape measure.

The ratio of height to thyromental distance refers to a ratio of height to the thyromental distance.

The ratio of neck circumference to thyromental distance refers to a ratio of the neck circumference to the thyromental distance.

The ratio of height to hyomental distance refers to a ratio of the height to the hyomental distance.
2) conducting an airway opening experiment on the patient corresponding to each group of the independent variable data, collecting glottis exposure images of the patient respectively at three preselected posture angles being 90°, 95°, and 100° in the experiment, performing laryngoscope exposure grading (Cormack-Lehane grade, CL grade) on a glottis exposure state in the images, and selecting an optimal posture angle making the patient be in an optimal glottis exposure state from the three preselected posture angles according to a laryngoscope exposure grading situation; and saving the optimal posture angle and the corresponding independent variable data.

A standard of the laryngoscope exposure grading (Cormack-Lehane grade, CL grade) is as follows:
grade I is that a glottis is completely exposed, and an anterior end and a posterior end of a rima glottidis are visible (namely, an anterior commissure and a posterior commissure are visible); grade II is that the glottis is partially exposed, and only the posterior end of the rima glottidis is visible; grade III is that only a tip of an epiglottis or the epiglottis is visible, but the glottis cannot be exposed; and grade IV is that neither the glottis nor the epiglottis can be exposed.

A method for selecting the optimal posture angle making the patient be in the optimal glottis exposure state from the three preselected posture angles according to the laryngoscope exposure grading situation includes:
according to the laryngoscope exposure grading situation of glottis exposure, if there is a preselected posture angle with a laryngoscope exposure grade below grade II, taking a preselected posture angle making the laryngoscope exposure grade lowest as the optimal posture angle, and if there are two or more aforementioned preselected posture angles, selecting a preselected posture angle with a minimum angle value as the optimal posture angle.

if laryngoscope exposure grades corresponding to the three preselected posture angles are all above grade III, an optimal posture angle corresponding to the group of independent variable data does not exist, and deleting the group of independent variable data.
3) performing an assignment processing on count independent variable data in each group of the saved independent variable data, and performing an original value retention processing on measurement independent variable data in each group of the saved independent variable data. In the example, gender, body mass index, modified Mallampati score, and upper lip bite test grade in the independent variable data are count independent variable data, the rest are measurement independent variable data, and the original value retention processing is performed on the measurement independent variable data in each group of the independent variable data.

An assignment processing is performed on the optimal posture angle corresponding to each group of the saved independent variable data, an assignment of the optimal posture angle serves as an outcome dependent variable, then data subjected to aforementioned processing are input into a statistical package for social sciences (Statistical Package for the Social Sciences, spss) to perform a single-factor analysis on various independent variables, and independent variables with statistical differences are screened out according to a single-factor analysis result.

The following table is an assignment table for the count independent variable data and outcome dependent variable data:

| Variable | Assignment |
|---|---|
| Gender | Female = 0, Male = 1 |
| BMI(kg/m2) | 18.5 to 23.9 = 1, |
| | ≤18.5 = 2, |
| | 24 to 27.9 = 3, |
| | ≥28 = 4 |
| Modified | <grade III = 0, |
| Mallampati score | ≥grade III = 1 |
| Upper lip bite test | grade I = 1, |
| grade | grade II = 2, |
| | grade III = 3 |
| Outcome dependent | posture angle 90° = 1, |
| variable | posture angle 95° = 2, |
| | posture angle 100° = 3 |

The following table is a baseline data table of the research subjects in the embodiment:

| Variable | Total (n = 769) |
|---|---|
| Age (years) | 60.0 |
| | (53.0, |
| | 69.0) |
| Gender | |
| Male | 491 |
| | (63.8%) |
| Female | 278 |
| | (36.2%) |
| BMI (kg/m2) | |
| ≤18.5 | 77 |
| | (10.0%) |
| 18.5 to 23.9 | 446 |
| | (58.0%) |
| 24 to 27.9 | 196 |
| | (25.5%) |
| ≥28 | 50 |
| | (6.5%) |
| Modified Mallampati score | |
| <grade III | 325 |
| | (42.3%) |
| ≥grade III | 444 |
| | (57.7%) |
| Upper lip bite test | |
| Grade I | 503 |
| | (65.4%) |
| Grade II | 214 |
| | (27.8%) |
| Grade III | 52 |
| | (6.8%) |
| Head and neck mobility (°) | 113.0 |
| | (102.0, |
| | 123.0) |
| Mouth opening degree (cm) | 4.3 |
| | (4.0, |
| | 4.5) |
| Neck circumference (cm) | 34.5 |
| | (32.0, |
| | 36.5) |
| Hyomental distance (cm) | 4.3 |
| | (4.0, |
| | 4.6) |
| Thyromental distance (cm) | 7.6 |
| | (7.2, |
| | 8.0) |
| Sternomental distance (cm) | 14.5 |
| | (13.6, |
| | 15.5) |
| Ratio of height to thyromental | 21.3 |
| distance | (20.0, |
| | 22.4) |
| Ratio of neck circumference | 4.47 ± 0.53 |
| to thyromental distance | |
| Ratio of height to hyomental | 37.8 |
| distance | (34.4, |
| | 41.3) |

The following table is a single-factor analysis table:

| | Instru- | Instru- | Instru- | | |
|---|---|---|---|---|---|
| | ment 90° | ment 95° | ment 100° | | |
| | group | group | group | | p- |
| Variable | (n = 354) | (n = 92) | (n = 92) | Statistic | value |
| Age | 61.0 | 58.5 | 57.5 | 8.100a | 0.017 |
| (years) | (54.0, | (50.0, | (51.3, | | |
| | 70.0) | 69.0) | 66.0) | | |
| Gender | | | | 2.294b | 0.318 |
| Male | 241 | 55 | 62 | | |
| | (68.1%) | (59.8%) | (67.4%) | | |
| Female | 113 | 37 | 30 | | |
| | (31.9%) | (40.2%) | (32.60%) | | |
| BMI | | | | 2.041a | 0.36 |
| (kg/m2) | | | | | |
| ≤18.5 | 31 | 10 | 13 | | |
| | (8.8%) | (10.9%) | (14.1%) | | |
| 18.5 to | 217 | 48 | 53 | | |
| 23.9 | (61.3%) | (52.2%) | (57.6%) | | |
| 24 to | 84 | 29 | 19 | | |
| 27.9 | (23.7%) | (31.5%) | (20.7%) | | |
| ≥28 | 22 | 5 | 7 | | |
| | (6.2%) | (5.4%) | (7.6%) | | |
| Modified | | | | 13.254b | 0.001 |
| Mallampati | | | | | |
| score | | | | | |
| <grade III | 173 | 34 | 27 | | |
| | (48.90%) | (37.0%) | (29.3%) | | |
| ≥grade III | 181 | 58 | 65 | | |
| | (51.1%) | (63.0%) | (70.7%) | | |
| Upper lip | | | | 0.287a | 0.867 |
| bite test | | | | | |
| Grade I | 265 | 66 | 68 | | |
| | (74.9%) | (71.7%) | (73.9%) | | |
| Grade II | 69 | 23 | 15 | | |
| | (19.5%) | (25.0%) | (16.3%) | | |
| Grade III | 20 | 3 | 9 | | |
| | (5.6%) | (3.3%) | (9.8%) | | |
| Head and | 115.0 | 115.0 | 115.0 | 1.205a | 0.548 |
| neck | (102.8, | (105.3, | (105.5, | | |
| mobility | 124.0) | 128.8) | 123.8) | | |
| (°) | | | | | |
| Mouth | 4.3 | 4.3 | 4.3 | 2.125a | 0.346 |
| opening | (4.0, | (4.0, | (4.0, | | |
| degree | 4.6) | 4.5) | 4.6) | | |
| (cm) | | | | | |
| Neck | 35.0 | 34.0 | 34.8 | 0.389a | 0.823 |
| circum- | (32.7, | (32.0, | (32.5, | | |
| ference | 36.5) | 37.0) | 36.6) | | |
| (cm) | | | | | |
| Hyo- | 4.5 | 4.0 | 4.0 | 43.874a | <0.001 |
| mental | (4.0, | (4.0, | (4.0, | | |
| distance | 5.0) | 4.5) | 4.5) | | |
| (cm) | | | | | |
| Thyro- | 8.0 | 7.4 | 7.2 | 126.513a | <0.001 |
| mental | (7.5, | (6.9, | (6.9, | | |
| distance | 8.5) | 7.6) | 7.5) | | |
| (cm) | | | | | |
| Sterno- | 15.0 | 14.5 | 14.3 | 29.109a | <0.001 |
| mental | (14.0, | (13.8, | (13.5, | | |
| distance | 16.0) | 15.0) | 15.0) | | |
| (cm) | | | | | |
| Ratio of | 20.8 | 22.3 | 22.6 | 128.843a | <0.001 |
| height | (19.5, | (21.3, | (21.4, | | |
| to thyro- | 21.7) | 22.8) | 23.3) | | |
| mental | | | | | |
| distance | | | | | |
| Ratio of | 4.3 | 4.7 | 4.8 | 65.829a | <0.001 |
| neck | (4.0, | (4.3, | (4.6, | | |
| circum | 4.7) | 5.1) | 5.1) | | |
| ference | | | | | |
| to thyro- | | | | | |
| mental | | | | | |
| distance | | | | | |
| Ratio of | 35.6 | 38.3 | 39.5 | 38.447a | <0.001 |
| height | (33.0, | (35.4, | (35.4, | | |
| to hyo- | 39.0) | 40.8) | 41.4) | | |
| mental | | | | | |
| distance | | | | | |

In the table: a = H, b = X².

4) selecting out independent variable data with statistical significance (namely P<0.05) in each group of the independent variable data according to an independent variable screening result, and configuring a data set by using the independent variable data with statistical significance and the outcome dependent variable. In the example, the independent variables with statistical differences screened out according to the single-factor analysis result are age, modified Mallampati score, hyomental distance, thyromental distance, sternomental distance, ratio of height to thyromental distance, ratio of neck circumference to thyromental distance, and ratio of height to hyomental distance.
5) preselecting several machine learning models configured to recommend the optimal airway opening posture angle, dividing the data set into a training set and a verification set, and respectively training and verifying each machine learning model by using data in the training set and the verification set. In the example, the preselected machine learning models include logistic regression, a decision tree, and a random forest.
6) aiming at various machine learning models obtained through training and verification, evaluating the various machine learning models by using average AUC values classified for the three preselected posture angles being 90°, 95°, and 100°, and selecting a machine learning model with a highest average AUC value as a prediction model for recommending the optimal airway opening posture angle.
\textcircled{1} An ROC curve is a curve drawn taking 1-specificity as an X-axis and sensitivity as a Y-axis. 1-specificity is represented by a False Positive Rate (FPR), sensitivity is represented by a True Positive Rate (TPR), and a differentiation capability of a classifier for positive and negative samples under different thresholds is reflected. An AUC value is defined as an area surrounded by the ROC curve and coordinate axes under the ROC curve, a value range is between 0.5 and 1, the closer the AUC is to 1, the better a model effect is; and when the AUC is equal to 0.5, the effect is worst, and there is no application value; an average calculation formula is as follows:

$${AUC}_{OVR} = \frac{1}{K} {\sum }_{i = 1}^{K} {AUC}_{i}$$

In the formula: AUC_{OVR} represents an average AUC value of a multi-classification model obtained based on a “one-vs-rest” strategy, and is used for comprehensively measuring performance of the multi-classification model; K: represents a total number of categories in a multi-classification problem, in the example k=3, namely representing 3 types of posture angles being 90°, 95°, and 100°;

$${\sum }_{i = 1}^{K} {AUC}_{i}$$

is a summation symbol, namely performing an accumulation operation on AUCᵢ from i=1 to i=K, AUCᵢ refers to an AUC value of a “one-vs-rest” binary classifier constructed aiming at an i-th category, namely an area under a curve corresponding to the binary classifier when an i-th category of samples is regarded as positive examples and all remaining categories of samples are regarded as negative examples;

$$\frac{1}{K}$$

represents an operation of calculating an average value on a result after examples; K AUCᵢ are summed, such that a binary classification AUC of each category has a same weight in a final calculation of AUC_{OVR}.

In the example, in addition to using the AUC value for evaluation on 3 kinds of machine learning models, indicators such as accuracy, macro-average precision, macro-average recall, and macro-average F1 value are also used to perform auxiliary evaluation on the 3 kinds of machine learning models. An explanation of evaluation indicators is as follows:
\textcircled{2} Accuracy: namely a proportion of all correctly classified samples to a total number of samples.
\textcircled{3} Precision: namely for each category (i), precision represents a proportion of samples truly belonging to a category among all samples predicted as the category.

Macro-average precision (Macro-Precision): namely treating each category equally, calculating an arithmetic average value of precisions of all categories, configured to measure average accuracy of a model predicting positive examples on various categories.
\textcircled{4} Recall: for each category (i), recall represents a proportion of being correctly predicted as a category among all samples actually belonging to the category.

Macro-average recall (Macro-Recall): taking an arithmetic average value of recalls of each category, measuring an average capability of the model correctly identifying positive examples on various categories.
\textcircled{5} F1 value: is a harmonic average value of the precision and the recall, and accuracy and completeness of the model are comprehensively considered.

Macro-average F1 value (Macro-F1): taking an arithmetic average value of F1 values of each category.

Evaluation results are shown in the following table:

| | | | Macro-average | | |
|---|---|---|---|---|---|
| | Aver- | | | | F1 |
| | age | | Precision | Recall | value |
| | AUC | Accu- | (macro- | (macro- | (macro- |
| Model | value | racy | average) | average) | average) |
| Logistic | 0.781 | 0.71 | 0.637 | 0.468 | 0.468 |
| regression | | | | | |
| (LR) | | | | | |
| Decision | 0.769 | 0.732 | 0.641 | 0.543 | 0.571 |
| tree | | | | | |
| (DT) | | | | | |
| Random | 0.912 | 0.804 | 0.856 | 0.636 | 0.693 |
| forest | | | | | |
| (RF) | | | | | |

In the example, the random forest is specifically selected as the prediction model for recommending the optimal airway opening posture angle. From the above evaluation results, an observation is made that the random forest performs best on three indicators of the average AUC value, the accuracy, and the macro-average.

Finally, the above examples are merely used to illustrate technical solutions of the present disclosure rather than limitation, although the present disclosure is described in detail with reference to the examples, those of ordinary skill in the art should understand that modifications or equivalent replacements may be made to the technical solutions of the present disclosure, without departing from a spirit and scope of the technical solutions of the present disclosure, and all the modifications or equivalent replacements should be covered within a scope of claims of the present disclosure.

What is claimed is:

1. A method for constructing a machine learning model for recommending an airway opening posture angle, comprising the following steps:
1) collecting independent variable data possibly affecting a glottis exposure effect when an airway of a patient is opened, each of the patients corresponding to a group of independent variable data;
2) conducting an airway opening experiment on the patient corresponding to each group of the independent variable data, collecting glottis exposure images of the patient respectively at three preselected posture angles being 90°, 95°, and 100° in the experiment, performing laryngoscope exposure grading on a glottis exposure state in the images, selecting an optimal posture angle making the patient be in an optimal glottis exposure state from the three preselected posture angles according to a laryngoscope exposure grading situation; and saving the optimal posture angle and the corresponding independent variable data;
3) performing an assignment processing on count independent variable data in each group of the saved independent variable data, performing an original value retention processing on measurement independent variable data in each group of the saved independent variable data, performing an assignment processing on the optimal posture angle corresponding to each group of the saved independent variable data, an assignment of the optimal posture angle serving as an outcome dependent variable, then inputting data subjected to aforementioned processing into a statistical package for social sciences to perform a single-factor analysis on various independent variables, and screening out independent variables with statistical differences according to a single-factor analysis result;
4) selecting out independent variable data with statistical significance in each group of the independent variable data according to an independent variable screening result, and configuring a data set by using the independent variable data with statistical significance and the outcome dependent variable;
5) preselecting several machine learning models configured to recommend the optimal airway opening posture angle, dividing the data set into a training set and a verification set, and respectively training and verifying each of the machine learning models by using data in the training set and the verification set; and
6) aiming at various machine learning models obtained through training and verification, evaluating the various machine learning models by using average AUC values classified for the three preselected posture angles being 90°, 95°, and 100°, and selecting a machine learning model with a highest average AUC value as a prediction model for recommending the optimal airway opening posture angle.

2. The method for constructing the machine learning model for recommending the airway opening posture angle according to claim 1, wherein in step 2), a method for selecting the optimal posture angle making the patient be in the optimal glottis exposure state from the three preselected posture angles according to the laryngoscope exposure grading situation comprises:
according to the laryngoscope exposure grading situation of glottis exposure, if there is a preselected posture angle with a laryngoscope exposure grade below grade II, taking a preselected posture angle making the laryngoscope exposure grade lowest as the optimal posture angle, and if there are two or more aforementioned preselected posture angles, selecting a preselected posture angle with a minimum angle value as the optimal posture angle; and
if laryngoscope exposure grades corresponding to the three preselected posture angles are all above grade III, an optimal posture angle corresponding to the group of independent variable data does not exist, and deleting the group of independent variable data.

3. The method for constructing the machine learning model for recommending the airway opening posture angle according to claim 1, wherein the independent variable data collected in step 1) comprises: gender, age, body mass index, modified Mallampati score, upper lip bite test grade, head and neck mobility, mouth opening degree, neck circumference, hyomental distance, thyromental distance, sternomental distance, ratio of height to thyromental distance, ratio of neck circumference to thyromental distance, and ratio of height to hyomental distance;
an acquisition method of the upper lip bite test grade is: the patient sits upright, a mandible is extended forward as much as possible, and lower incisors are used to bite an upper lip as much as possible, if an upper lip line is exceeded, a grade is grade I; if the lower incisors are lower than the upper lip line, the grade is grade II; and if the lower incisors cannot bite the upper lip, the grade is grade III;
the head and neck mobility refers to an angle formed by maximally flexing a neck forward to make a mental prominence lean against a sternal notch, and a maximal head back-tilt position;
the mouth opening degree refers to a distance between upper and lower central incisors when the patient is in a neutral sitting position and opens a mouth to a maximum extent;
the hyomental distance refers to a distance from an anterior border of a hyoid bone to a mental protuberance of the mandible;
the thyromental distance refers to a linear distance from a superior thyroid notch to a submental angle of the mandible when the patient is in a maximal head back-tilt state and the mouth is closed;
the sternomental distance refers to a linear distance from a suprasternal notch to the submental angle of the mandible when the patient closes the mouth and the head and neck are in a maximal back-extension position;
the ratio of height to thyromental distance refers to a ratio of height to the thyromental distance;
the ratio of neck circumference to thyromental distance refers to a ratio of the neck circumference to the thyromental distance; and
the ratio of height to hyomental distance refers to a ratio of the height to the hyomental distance.

4. The method for constructing the machine learning model for recommending the airway opening posture angle according to claim 3, wherein an acquisition method of the modified Mallampati score is: the patient takes a sitting position, a head is in a neutral position, the mouth is opened and a tongue is extended to a maximum extent as much as possible without phonation, and grading is performed according to visibility of an oropharyngeal structure; grade I: a soft palate, a palatopharyngeal arch, and a uvula are visible; grade II: the soft palate and the palatopharyngeal arch are visible, but the uvula is partially blocked by a tongue base; grade III: only the soft palate is visible; and grade IV: the soft palate is not visible.

5. The method for constructing the machine learning model for recommending the airway opening posture angle according to claim 3, wherein in step 3), the independent variables with statistical differences screened out according to the single-factor analysis result are age, modified Mallampati score, hyomental distance, thyromental distance, sternomental distance, ratio of height to thyromental distance, ratio of neck circumference to thyromental distance, and height hyomental distance.

6. The method for constructing the machine learning model for recommending the airway opening posture angle according to claim 5, wherein the machine learning models preselected in step 5) comprise logistic regression, a decision tree, and a random forest.

7. The method for constructing the machine learning model for recommending the airway opening posture angle according to claim 6, wherein in step 6), the random forest is selected as the prediction model for recommending the optimal airway opening posture angle.
